(19) United States
(12) Patent Application Publication
(10) Pub. No.: US 2005/0203057 A1
(43) Pub. Date: Sep. 15, 2005

(54) Treatment of cancers

(76) Inventor: Yash Sharma, Chantilly, VA (US)

Correspondence Address: BAKER & MCKENZIE LLP, 711 LOUISIANA, SUITE 3400, HOUSTON, TX, 77002-2716, US

(21) Appl. No.: 11/033,809

(22) Filed: Jan. 13, 2005

Related U.S. Application Data
(63) Continuation-in-part of application No. 09/474,677, filed on Dec. 9, 1999.
Application No. 09/474,677 is a continuation-in-part of application No. 09/015,830, filed on Jan. 29, 1998.
(60) Provisional application No. 60/632,982, filed on Dec. 6, 2004.
(60) Provisional application No. 60/114,540, filed on Dec. 29, 1998.

(51) Int. Cl. A61K 31/739; A61K 9/68

(52) U.S. Cl. 514/54; 424/440

(57) ABSTRACT

Methods for treating a cancer in a subject are described that include administering N-glycolylneuraminic acid or a derivative thereof to the subject.

### CROSS REFERENCE TO RELATED APPLICATIONS

This application claims priority to U.S. Provisional Patent Application No. 60/632,982, filed Dec. 6, 2004, and is a continuation-in-part of U.S. patent application Ser. No. 09/474,677, filed Dec. 29, 1999, which claims priority to U.S. Provisional Application No. 60/114,540, filed Dec. 29, 1998, and is a continuation-in-part of U.S. patent application Ser. No. 09/015,830, filed Jan. 29, 1998, the disclosures of which are incorporated by reference in their entirety.

### BACKGROUND OF THE INVENTION

Cancer is a group of many related diseases that are generally named for the organ or type of cell in which they begin. Normally, cells grow and divide to produce more cells only when cells are needed by the body, and old cells die, and new cells take their place. Cancerous cells keep dividing, when new cells are not needed, and often old cells do not die when they should. In certain types of cancer, the cancerous cells form a mass of tissue, called a malignant growth or tumor. Cancer cells can spread from the original cancer site to other parts of the body through metastasis.

The most common types of cancer diagnosed in the United States are bladder cancer, breast cancer, colo-rectal cancer, endometrial cancer, kidney cancer, leukemia, lung cancer, melanoma, non-Hodgkin's lymphoma, ovarian cancer, pancreatic cancer, prostate cancer, and skin cancer (non-melanoma), with non-melanoma skin cancer being the most commonly diagnosed cancer in the U.S.

Treatments for cancer include: surgery, radiation therapy, chemotherapy, hormone therapy, and biological therapy. Cancer treatments can have harmful effects on healthy cells in the body, and because treatment damages healthy cells and tissues, they often cause unpleasant side effects. Surgery is one therapy involving removing the cancer; the surgeon may also remove some of the surrounding tissue and lymph nodes near the tumor. The side effects of surgery depend on many factors, including the size and location of the tumor, the type of operation, and the patient's general health.

Radiation therapy uses high-energy radiation to kill cancer cells. Radiation may be used alone, or along with other types of treatment. The side effects of radiation therapy depend on the treatment dose and the part of the body that is treated. Patients are likely to become extremely tired during radiation therapy, especially in the later weeks of treatment. With external radiation, there may be permanent darkening or “bronzing” of the skin in the treated area. In addition, it is common to have temporary hair loss in the treated area and for the skin to become red, dry, tender, and itchy. Radiation therapy also may cause a decrease in the number of white blood cells that help protect the body against infection.

Chemotherapy is the use of drugs to kill cancer cells. One drug or a combination of drugs may be used. Chemotherapy may be the only kind of treatment a patient needs, or it may be combined with other forms of treatment. The side effects of chemotherapy depend mainly on the drugs and the doses the patient receives. Generally, anticancer drugs affect cells that divide rapidly. In addition to cancer cells, these include blood cells, which fight infection, help the blood to clot, and carry oxygen to all parts of the body. When blood cells are affected, patients are more likely to get infections, may bruise or bleed easily, and may feel unusually weak and very tired. Rapidly dividing cells in hair roots and cells that line the digestive tract may also be affected. As a result, side effects may include loss of hair, poor appetite, nausea and vomiting, diarrhea, or mouth and lip sores. Some anticancer drugs can cause long-term side effects such as loss of fertility.

Hormone therapy is used against certain cancers that depend on hormones for their growth. Hormone therapy keeps cancer cells from getting or using the hormones they need. This treatment may include the use of drugs that stop the production of certain hormones or that change the way they work. Hormone therapy can cause a number of side effects. Patients may feel tired, have fluid retention, weight gain, hot flashes, nausea and vomiting, changes in appetite, and, in some cases, blood clots.

Biological therapy (also called immunotherapy) helps the body's immune system to fight disease or protects the body from some of the side effects of cancer treatment. Monoclonal antibodies, interferon, interleukin-2, and colony-stimulating factors are some types of biological therapy. The side effects caused by biological therapy vary with the specific treatment. In general, these treatments tend to cause flu-like symptoms, such as chills, fever, muscle aches, weakness, loss of appetite, nausea, vomiting, and diarrhea. Patients also may bleed or bruise easily, get a skin rash, or have swelling. These problems can be severe, but they go away after the treatment stops.

Bone marrow transplantation (BMT) or peripheral stem cell transplantation (PSCT) may also be used in cancer treatment. Both BMT and PSCT provide the patient with healthy stem cells. These replace stem cells that have been damaged or destroyed. Patients who have a BMT or PSCT face an increased risk of infection, bleeding, and other side effects due to the high doses of chemotherapy and/or radiation they receive. The most common side effects associated with the transplant itself are nausea and vomiting during the transplant, and chills and fever during the first day or so. In addition, graft-versus-host disease (GVHD) may occur in patients who receive bone marrow from a donor.

Leukemia and lymphoma are cancers that arise in blood-forming cells. Leukemia as a cancer type includes cases of acute lymphoblastic (or lymphoid) leukemia, chronic lymphocytic leukemia, acute myelogenous leukemia, chronic myelogenous (or myeloid) leukemia, and other leukemia types. More than 33,000 new cases of leukemia will have been diagnosed in the United States in 2004, with acute myelogenous leukemia being the most common type (approximately 12,000 new cases). Symptoms of leukemia may not appear for a long time. Such symptoms can include fevers or night sweats, frequent infections, feeling weak or tired, headache, bleeding and bruising easily, pain in the bones or joints, swelling or discomfort in the abdomen (from an enlarged spleen), swollen lymph nodes, and weight loss.

In acute leukemia, symptoms appear and get worse quickly. Symptoms of acute leukemia can include vomiting, confusion, loss of muscle control, and seizures. Some patients develop sores in the eyes or on the skin. Leukemia also can affect the digestive tract, kidneys, lungs, or other parts of the body. Depending on the type and extent of the disease, patients may have chemotherapy, biological therapy, radiation therapy, or bone marrow transplantation. If the patient's spleen is enlarged, the doctor may suggest surgery to remove it. In addition to anticancer therapy, people with leukemia may have treatment to control pain and other symptoms of the cancer, to relieve the side effects of therapy, or to ease emotional problems.

Clearly both the side effects of cancers, as well as, cancer treatments can in some cases be debilitating, painful, and invasive. Additional treatment options for cancer, especially leukemia, are urgently needed. Safe, and orally bioavailable anti-cancer agents are desirable.

N-glycolylneuraminic acid (Neu5Gc) is a cell surface sialic acid that is immunogenic in humans. Sialic acids, such as Neu5Gc, are N-acyl derivatives of neuraminic acid. Sialic acids occur in many polysaccharides, glycoproteins, and glycolipids in both animals and bacteria. Neu5Gc is widespread throughout the animal kingdom. However, due to a genetic mutation in the human gene that hydrolyzes acetyl neuraminic acids, Neu5Gc is nearly absent on the cell surface of human cells.

### SUMMARY OF THE INVENTION

Certain aspects of the present invention are directed to methods for treating a human patient having a cancer. The cancer can be a bladder cancer, a breast cancer, a colo-rectal cancer, a endometrial cancer, a kidney cancer, a leukemia, a lung cancer, a melanoma, a non-Hodgkin's lymphoma, a ovarian cancer, a pancreatic cancer, a prostate cancer, a hepatocellular carcinoma or a non-melanoma skin cancer, among others.

The methods can involve administering a composition to a patient. The composition can have at least one of a N-glycolylneuraminic acid or a derivative thereof, and at least one pharmaceutically acceptable excipient, among other components. The administration of the composition can be at least sufficient to slow progression of the cancer.

In some embodiments, the methods can further involve determining a baseline TNFα level of a patient prior to administering the composition, and determining a post-administration TNFα level. The administration can involve a dosing regimen of the composition such that the post-administration TNFα level is less than about 95% of the baseline TNFα level. In some embodiments the post-administration level is less than about 90% of the baseline, and in others the level is less than about 60%.

In certain aspects of the present invention, at least some of the N-glycolylneuraminic acid or derivative thereof in the composition is bound, and the composition can further have a catalytic amount of at least one pharmaceutically acceptable acid or a salt thereof. The composition used in methods of present invention can be administered intravenously, subcutaneously, by inhalation, transdermally, or bucally, among others. In certain embodiments, between about 0.1 mg and 1000 mg of the composition can be administered to a patient per day. In some aspects of the invention, the composition can be in the form of a tablet, a lozenge, a sucker, a semi-soft candy, a gum, a gel, a paste, a mouthwash, or a film, among others.

Other features and advantages of the invention will be apparent from the following detailed description, and from the claims.

### BRIEF DESCRIPTION OF THE DRAWINGS

FIG. 1 is a chromatograph that depicts the HPLC Profile of LUKOR (C18 column; 0.1% TFA/water and a 0-100% ACN gradient; UV detection A280).

### DETAILED DESCRIPTION

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

As used herein, “buccal administration” refers to oral administration of a composition to a patient that is held in the mouth and is used to deliver N-glycolylneuraminic acid or a derivative thereof into a patient's body. Certain compositions of the present invention can, for example, be held in the patient's mouth and sucked, to release N-glycolylneuraminic acid or a derivative thereof into the buccal cavity.

Regarding “N-glycolylneuraminic acid or derivatives thereof,” N-glycolylneuraminic acid (C₁₁H₁₉NO₁₀, MW 325.3) is the hydroxylated derivative of N-acetylneuraminic acid (sialic acid). As used herein, “derivative” refers to a compound that is similar in structure to N-glycolylneuraminic acid, such as the compounds described in U.S. Pat. Nos. 4,774,326 and 4,774,327, and N-linked glycans, as well as compounds such as glucosamine 1-acetylneuraminic acid and mannosamine acetylneuraminic acid. Additional non-limiting examples of derivatives include phosphorylated or sulfated N-glycolylneuraminic acid, N-glycolylneuraminic acid salts, O-glycolylneuraminic acid, as well as other substituted N-glycolylneuraminic acid compounds. Attempts have been made to produce Neu5Gc through chemical synthesis, but these efforts have met with limited success. As an alternative to chemical synthesis, Neu5Gc can be recovered from biological samples, especially animal tissues. It can be more cost effective to use unpurified or partially purified biological extracts.

“Bound N-glycolylneuraminic acid or derivatives thereof” refers to N-glycolylneuraminic acid (Neu5Gc) or derivatives thereof that are chemically bound to glycolipids, glycoproteins, other glycoconjugates, or phospholipids. The Neu5Gc or derivatives thereof present in certain non-human animals is often found bound to glycoproteins, glycolipids, or other glycoconjugates, or phospholipids. However, in order for Neu5Gc or derivatives thereof to be more effective in producing an immune response in humans, it may be preferable that they be introduced into the body in a chemically free form. Bound Neu5Gc or derivatives thereof can be isolated from phospholipids or glycoconjugates using nontoxic agents to break their bonds with these compounds.

As used herein, “pharmaceutically acceptable” refers to substances that are generally regarded as safe for introduction into the human body.

An “excipient” refers to an inert substance used in compositions of the present invention to make them easier to administer.

A “therapeutic agent” refers to compounds that are used to treat specific diseases or medical conditions.

“Essential oil” refers to a natural oil with a distinctive scent secreted by the glands of certain aromatic plants having terpenes as the major component. Examples of essential oils include, but are not limited to, citrus oils, flower oils (e.g., rose and jasmine), and oil of cloves.

“Nonnutritive sweetener” refers to a synthetic or natural substance whose sweetness is higher than or comparable to sucrose, and which may have properties such as reduced cariogenicity, health benefits for diabetics, or reduced caloric value compared to sugars.

“Catalytic amount” refers to having a sufficient amount of an acid or salt thereof in a composition of the present invention, such that it may act as a catalyst for hydrolysis. The hydrolysis results in the freeing of at least some N-glycolylneuramic acid or a derivative thereof from being bound to glycoconjugates or phospholipids.

“Staging” refers to performing exams and tests to learn the extent of the cancer within the body of a patient, especially whether the disease has spread from the original site to other parts of the body. In some cases, the cancer of patients can be staged using the TNM (Tumor/Lymph nodes/Metastisis) system or their cancer can be described as being in stage 0-IV. In TNM staging a number is added to each letter (e.g., T, N, or M) to indicate the size or extent of the tumor and the extent of spread. In a different staging system, stage 0 is early cancer that is present only in the layer of cells in which it began, stages I-III indicate a more extensive disease state (greater tumor size and/or spread of the cancer to nearby lymph nodes and/or organs adjacent to the primary tumor), and stage IV indicates the cancer has spread to another organ. Some staging systems cover many types of cancer and others focus on a particular type. Cancers of the brain and spinal cord are classified according to their cell type and grade. Different staging systems are also used for many cancers of the blood or bone marrow. The common elements considered in most staging systems are: location of the primary tumor or cancer cells, tumor size and number of tumors and/or number of cells affected, lymph node involvement, cell type and tumor grade, and metastasis.

“TNFα-sensitive cancers” refers to cancers that respond positively to reducing the Tumor Necrosis Factor alpha (TNFα) levels of a patient.

“Growth rate of cancer” can be measured by determining carcinogen embryonic antigen (CEA) levels for breast cancer. Carcinogen or cancer antigen (CA) levels can be followed for other types of cancer. If levels of CEA or CA increase then the growth of the cancer is perceived, and these measurements can be made over time. As the levels drop the growth rate of the cancer decreases, while if the levels remain the same, the cancer is thought not to be progressing.

“Progression of cancer” relates to staging and refers to progression from cancer in single cell type (tissue) to increasing number of cells affected to spreading to lymph nodes to spreading to other organs. Regression is moving back within or from a stage in the progression. In “remission” the patient shows no symptoms of the cancer and cancerous cells are no longer detected.

Methods of the present invention for treating a human patient having a cancer can involve administering a composition to a patient. The composition can have at least one of a N-glycolylneuraminic acid or a derivative thereof, and at least one pharmaceutically acceptable excipient, among other components. Administration of the composition can be such that it is at least sufficient to slow progression of the cancer. In some embodiments, the composition may be administered to the patient in addition to at least one other cancer treatment or a medication to alleviate or reduce a symptom of the cancer or the other cancer treatment.

In certain embodiments of the present invention, methods may involve staging the cancer in the patient prior to administering the composition, and staging the cancer post-administration of the composition. In some embodiments, administration may involve a dosing regimen of the composition such that the post-administration staging is the same or less severe than prior to administration. In certain embodiments, it may be determined that the cancer does not progress after administration of the composition. In some embodiments, it may be found that the cancer regresses after administration of the composition.

Methods of the present may involve determining a baseline growth rate of the cancer prior to administering the composition, and determining a post-administration growth rate of the cancer. The administration of the composition may be done with a dosing regimen such that the post-administration growth rate may be less than 90%, 60%, or 40% of the baseline growth rate, in some embodiments.

Certain methods of the present invention may involve determining a baseline TNFα level of a patient prior to administering the composition, and determining a post-administration TNFα level. In some embodiments, the administration may involve a dosing regimen of the composition such that the post-administration TNFα level is less than 97%, 60%, or 40% of the baseline TNFα level.

Not to be bound by theory, but it is known that normal humans have variable amounts of circulating IgA, IgM, and IgG antibodies against Neu5Gc or derivatives thereof. Humans having the highest levels of such antibodies, the levels may be comparable to levels of antibodies to anti-α-galactose xenoreactive antibodies known in the art. Humans may absorb and metabolically incorporate a nonhuman dietary component in certain foods enriched in the antigenic component, even while generating xenoreactive, and potentially autoreactive, antibodies against the same antigenic molecule, such as Neu5Gc. Since Neu5Gc may be incorporated into cancer cells at a high rate, the xenoreactive antibodies may attack the cancerous cells, causing a slowing of progression of the cancer or regression of the cancer. Furthermore, it has been demonstrated that Neu5Gc may reduce TNFα levels in patients.

In certain aspects of the invention a patient may have a bladder cancer, a breast cancer, a colo-rectal cancer, a endometrial cancer, a kidney cancer, a leukemia, a lung cancer, a melanoma, a non-Hodgkin's lymphoma, a ovarian cancer, a pancreatic cancer, a prostate cancer, a hepatocellular carcinoma or a non-melanoma skin cancer, among others. In some embodiments, the cancer is a TNFα-sensitive cancer, such as leukemia, among others. The cancer may be leukemia or a hepatocellular carcinoma in certain embodiments. In some aspects of the invention the cancer may be leukemia. The cancer may be acute lymphoblastic leukemia, chronic lymphocytic leukemia, acute myelogenous leukemia, or chronic myelogenous, among others. A patient may have a TNFα-sensitive cancer, and a TNFα level in the patient is lower in the patient post-administration of the composition than before administration of the composition, in some embodiments. In certain embodiments, a patient may have leukemia, and a TNFα level in the patient is lower in the patient post-administration of the composition than before administration of the composition.

In some embodiments, the composition may have between about 0.002 wt % and 20 wt % of the N-glycolylneuraminic acid or the derivative thereof. In other embodiments, the composition may have between about 0.1 wt % and 20 wt % of the N-glycolylneuraminic acid or the derivative thereof, and in some, between about 0.1 wt % and 10 wt % of the N-glycolylneuraminic acid or the derivative thereof. In certain embodiments, the composition may have a derivative of N-glycolylneuraminic acid, and the derivative may be a phosphorylated N-glycolylneuraminic acid or a sulfated N-glycolylneuraminic acid, among others known in the art. In certain aspects of the present invention the composition may have synthetic N-glycolylneuraminic acid or a derivative thereof. The composition may have bound or free N-glycolylneuraminic acid or a derivative thereof that has been extracted from a biological sample in some aspects of the invention. In certain embodiments, the composition may include a biological sample that comprises the N-glycolylneuraminic acid or a derivative thereof. In particular, N-glycolylneuraminic acid may be obtained from or be a component of at least one of a sea cucumber extract; a peripheral blood mononuclear cell extract of a non-human animal (e.g., a pig or baboon PBMC extract); a submaxillary gland extract of a non-human animal (e.g., a equine, bovine or porcine submaxillary gland extract); an extract from meat or meat fat (e.g., beef, pork, lamb, and poultry) consumed by humans; a milk, butter or cheese extract (e.g., bovine, goat, and sheep milks and cheeses); a fish extract (e.g., cod, tuna, and salmon extracts), a starfish extract, a shark extract, a crocodile extract, or a sea urchin extract, among others known in the art. In certain aspects of the present invention, a sea cucumber extract, a pig submaxillary gland extract, a salmon extract, a milk, butter, or cheese extract, a lamb extract, a pork extract, a beef extract, or a beef fat extract may be used as the source of bound or free N-glycolylneuraminic acid or derivatives thereof in the composition. In some embodiments of the present invention, a sea cucumber extract, a meat extract, a meat fat extract, or a goat cheese extract may be used. In some aspects of the present invention, a sea cucumber extract may be used. Extracts comprising N-glycolylneuraminic acid or derivatives thereof may be purchased commercially from, for example, Sigma Aldrich as a porcine submaxillary gland extract. (PNAS 100, October, 2003)

In some embodiments, the composition that is administered to patient having a cancer may have at least some N-glycolylneuraminic acid or derivative thereof that is bound, and the composition may further have a catalytic amount of at least one pharmaceutically acceptable acid or a salt thereof. Certain compositions of the present invention may have between about 0.01 wt % and 50 wt % of an acid or a salt thereof. In certain aspects of the present invention, the compositions may have between about 0.01 wt % and 5 wt % of an acid or a salt thereof. In some embodiments, the compositions may have between about 0.1 wt % and 5 wt % of an acid or a salt thereof. Examples of acids that may be used in the present invention include: salisalic acid, glycolic acid, phosphoric acid, pentathoic acid, and ascorbic acid, among others known in the art. The composition may comprise salts of such acids.

In certain aspects, the composition may have ascorbic acid or salt thereof. Examples of ascorbic acid salts that could be used in compositions of the present invention include mono-, di-, and tri-sodium citrate salts of ascorbic acid, among others. Such acids or salts thereof are available commercially from Sigma Aldrich, St. Louis, Mo. Certain compositions of the present invention may comprise between about 0.002 wt % and 20 wt % ascorbic acid or a salt thereof. In some embodiments, the compositions comprise between about 0.01 wt % and 5 wt % ascorbic acid or a salt thereof. In certain embodiments, the compositions comprise between about 0.1 wt % and 5 wt % ascorbic acid or a salt thereof.

Compositions employed in the present invention may have a pharmaceutically acceptable excipient and may be, for example, mannitol, cyclodextrins and their derivatives magnesium stearate, calcium carbonate, sodium carbonate, lactose, D-mannitol, calcium phosphate, sucrose, sodium chloride, glucose, starch, kaolin, cellulosic materials, anhydrous calcium secondary phosphate, light anhydrous silicic acid, partly pregelatinized starch, acacia powder, gum arabic, sorbitol, corn starch, or alginic acid, among others known in the art. In some embodiments other components listed below may be used as an excipient (e.g., a lubricant such as magnesium stearate).

According to some embodiments, the excipient may serve more than one role in the composition. For example, mannitol may function as both a nonnutritive sweetener and an excipient. Similarly, the excipient may serve as a flavorant, buffering agent, lubricant, or other component of the composition. The excipient may be present in an amount less than about 90 wt % by weight (wt %), in some embodiments in an amount less than about 80 wt %, and in certain embodiments, in an amount less than about 50 wt % in a composition of the present invention.

Compositions of the present invention may be administered by a number of different methods known in the art. For example, the composition may be administered intravenously, subcutaneously, by inhalation, transdermally, or buccally. In certain embodiments in which a composition is administered buccally, the composition may be at least partially dissolved by saliva in the patient's mouth. In some embodiments involving buccal administration, at least some of the N-glycolylneuraminic acid or derivative thereof may be bound, and the composition may have a catalytic amount of at least one pharmaceutically acceptable acid or a salt thereof, and at least some of the acid or salt thereof may be dissolved by saliva in the patient's mouth. In certain embodiments, compositions for buccal administration of the present invention may have at least one extract having at least one bound N-glycolylneuraminic acid or a derivative thereof, at least one of ascorbic acid or a salt thereof, and at least one pharmaceutically acceptable excipient. In certain embodiments, the composition may have between about 0.002 wt % and 20 wt % bound N-glycolylneuraminic acid or a derivative thereof, and between about 0.02 wt % and 50 wt % ascorbic acid or a salt thereof.

The composition may be in the form of a tablet, a lozenge, a sucker, a semi-soft candy, a gum, a gel, a paste, a mouthwash, or a film, in certain embodiments of the present invention. In some embodiments, compositions of the present invention may further have at least one pharmaceutically acceptable component selected from a coloring agent, a polypeptide, a lubricant, a coating, a sweetener, a flavoring, an antibacterial agent, a taste modifier, a preservative, a disintegrator, a disintegration-preventor, a binder, an antioxidant, a dietary supplement, an antiblocking agent, an antisticking agent, an absorption promoter, absorption-adsorption carriers or a therapeutic agent, among others known in art. Such components when present in certain embodiments do not interfere with release and absorption of N-glycolylneuraminic acid. As with the excipient, some of these components of a composition may serve in more than one role.

Examples of disintegrators, include dry starch, alginic acid, agar powder, crosslinked polyvinyl pyrrolidone, crosslinked sodium carboxymethylcellulose, L-hydroxypropylcellulose, calcium carboxymethylcellulose, and sodium starch glycolate, among others. Examples of disintegration-preventors, are stearyl alcohol, stearic acid, cacao butter, and hydrogenated oil, among others. Binders such as gelatin, crystalline cellulose, simple syrup, sucrose, glucose solution, starch solution, polyvinyl alcohol, polyvinyl ether, polyvinylpyrrolidone, carboxymethylcellulose, shellac, methylcellulose, ethylcellulose, sodium alginate, gum arabic, hydroxypropylmethylcellulose, hydroxypropylcellulose, water, D-mannitol, dextrin, ethanol, starch, gelatin, and acacia, among others may be used in certain compositions.

Antiblocking and antisticking agents such as aluminum silicate, calcium hydrogen phosphate, magnesium oxide, talc, and silicic acid anhydride, among others, may be used in certain compositions of the present invention. Lubricants such as magnesium stearate, calcium stearate, stearic acid, carnauba wax, light silicic acid anhydride, aluminum silicate, magnesium silicate, hardened oil, hardened vegetable oil derivatives, colloidal silica, sesame oil, bleached bees wax, titanium oxide, dry aluminum hydroxide gel, calcium hydrogen phosphate, sodium lauryl sulfate, polyethylene glycol, and talc, among others, may be used in certain compositions of the present invention. In certain embodiments, the lubricant may be present in an amount between about 0.1 and 25 wt %, in certain embodiments in an amount between about 0.1 and 10 wt %, and in certain aspects of the invention in an amount between about 0.1 and 5 wt % of the inventive composition. Examples of absorption promoters that may be used in certain embodiments of the present invention include quaternary ammonium salts, sodium lauryl sulfate, urea, and enzymes, among others. Examples of absorption-adsorption carriers are starch, lactose, kaolin, bentonite, silicic acid anhydride, hydrated silicon dioxide, magnesium metasilicate-aluminate, and colloidal silicic acid, among others.

Further, if desired, a tablet, a semi-soft candy, a gum, a sucker, or a lozenge may be coated. The coating may be made with sugar, or gelatin, among others compounds. The coating may comprise hydroxypropylmethylcellulose, ethylcellulose, hydroxymethylcellulose, hydroxypropylcellulose, polyoxyethylene glycol, Tween 80, Pluronic F68, cellulose acetate phthalate, hydroxypropylmethylcellulose phthalate, hydroxymethylcellulose acetate succinate, Eudragit (methacrylic acid/acrylic acid copolymer, manufactured by Rohm and Haas, DE), or pigment (e.g., iron oxide red, titanium dioxide, et.), among other coating components known in the art.

Flavorings that may be used in the present invention include sweeteners, especially non-nutritive sweeteners. Examples of flavorings that could be used in certain embodiments, include acacia or tragacanth, among others. Certain compositions of the present invention may comprise more than one sweetener. In certain embodiments, the flavoring comprises a nonnutritive sweetener that is noncariogenic. The cariogenicity of a substance is dependent upon its susceptibility to fermentation by Streptococcus mutans and other oral microorganisms. Dental researchers have long recognized that fermentable sweeteners such as sucrose, glucose, starch, and corn syrup are cariogenic or cavity causing. Examples of nonnutritive sweeteners that may be used in compositions of the present invention include: saccharin, invert sugar, cyclamate, palantinose, aspartame, xylitol, acesulfame, sorbitol, monellin, mannitol, meohesperidine, maltitol, and palatinit, among others. In certain compositions of the present invention, the nonnutritive sweetener may be present in an amount between about 50 and 90 wt %, in certain embodiments in an amount between about 70 and 90 wt %, and in some embodiments in an amount between about 80 and 90 wt %.

Other flavorings that may be used in compositions of the present invention include a candy taste, such as chocolate, orange, vanilla, and the like; essential oils such as peppermint, spearmint and the like; or other flavor, such as anis seed, eucalyptus, 1-menthol, carvone, and anethole, among others known in the art. Both individual and mixed flavors are contemplated. The flavorings are generally utilized in amounts that will vary depending upon the individual flavor, and may, for example, range in amounts of about 0. 1% to about 6% by weight of the final composition.

In certain embodiments, fluoride, and more particularly sodium monofluorophosphate or sodium fluoride may be incorporated into a composition of the present invention, especially one having a nonnutritive sweetener, such as xylitol.

The coloring agents useful in the present invention include pigments which may be incorporated in amounts of up to about 2% by weight of the composition. Also, the coloring agents may include other dyes suitable for food, drug and cosmetic applications (i.e., FD&C dyes) and the like. The materials acceptable for the foregoing spectrum of use are, in some embodiments, water-soluble. Illustrative examples include the indigo dye known as FD&C Blue No. 2, which is the disodium salt of 5,5-indigotindisulfonic acid, FD&C Green No. 1, which is a triphenylmethane dye and is the monosodium salt of 4-[4-N-ethyl-p-sulfobenzyl amino)diphenyl-methylene]-[1-(N-ethyl--N-p-sulfoniumbenzyl)-2,5-cyclohexadienimine]. Other FD&C and D&C colorants useful in the present invention and their corresponding chemical structures may be found in the Kirk-Othmer Encyclopedia of Chemical Technology, 3rd Edition, in Volume 6, at pages 561-595.

The amount of N-glycolylneuraminic acid or a derivative thereof effective for treating a cancer in a subject may vary, depending on a number of factors, including the amount of the composition in individually prepared doses (e.g., tablets) conveniently available, the chemical characteristics of the compounds employed, the formulation of the compound excipients and the route of administration. The optimal dosage of N-glycolylneuraminic acid to be administered also may depend on such variables as the overall health status of the particular patient and the relative biological efficacy of the compound (e.g., N-glycolylneuraminic acid or derivatives thereof) selected. Compositions of the present invention may have between about 0.002 wt % and 20 wt % bound or free N-glycolylneuraminic acid or a derivative thereof. In some embodiments, the compositions may have between about 0.01 wt % and 5 wt % bound or free N-glycolylneuraminic acid or a derivative thereof. In certain embodiments, the compositions may have between about 0.01 wt % and 2.5 wt % bound or free N-glycolylneuraminic acid or a derivative thereof.

Dosing regimens that may be used in the present invention include administering the composition at least once per day. In certain embodiments, the dosing regimen involves administering the composition at least three times per day. In some embodiments, the dosing regimen involves administering the composition at least four times per day. In addition, delayed release formulations of the composition may be used such that administrations are less frequent. In certain embodiments, a physician may prescribe the proper dosages and dosing regimen. The composition may be, in certain embodiments, in unit dosage form. In such form the composition is subdivided into unit doses containing appropriate quantities of the active component. The unit dosage form may be a packaged preparation, the package containing discrete quantities of preparation, such as packeted tablets or lozenges. In certain embodiments, the unit dosage form may be a tablet or lozenge itself, or it may be the appropriate number of any of these in packaged form.

The dosing regimen in certain aspects of the present invention may involve administering to the patient between about 0.1 mg and 1000 mg of the composition per day. In some embodiments, between about 0.2 mg and 100 mg of the composition may be administered per day, and in certain embodiments, between about 0.2 mg and 80 mg of the composition may be administered per day.

### EXAMPLES

### Example 1

### Isolation of a Small Molecule from Baboon Blood

Peripheral blood monocytes (PBMC) were isolated from whole baboon blood using Ficoll-Hypaque density gradient centrifugation or from PBMCs further expanded in tissue culture following activation with phytohemagglutinin-P (PHA-P) and growth in medium containing interleukin-2 (IL-2). In either case, the PBMCs first were washed 3 times with sterile phosphate-buffered saline (PBS) and pelleted by centrifugation. The cell pellet then was lysed by resuspension in sterile H₂O and held for 96 hours at 4° C. Proteins and nucleic acids were precipitated from the extract and the remaining components in the extract were stabilized using 10% (v/v) calcium phosphate buffer (pH 7.4) containing 0.01% calcium chloride and 0.001% ascorbic acid. The solution was clarified by centrifugation followed by filtration through a 0.22 μm filter. This final filtrate represented a 1:50 dilution of the initial cell lysate and is hereafter referred to as LUKOR. In some instances, the LUKOR preparation was sterilized by Cobalt radiation at 2.5 mRADs for 3 hours (Neutron Products, Inc. Gaithersburg, Md.).

A series of in vitro and in vivo toxicology studies were conducted to evaluate the potential adverse effects of LUKOR on human blood cells, blood clotting factors, and in rats administered intravenous LUKOR repeatedly over 28 days. Freshly drawn heparinized whole blood (1 ml) was diluted 1:4 in PBS (pH 7.4) and 0.1 ml of LUKOR (freshly prepared) was added per ml of diluted whole blood. Two aliquots of the blood were maintained at 4° C. for 21 and 42 days. Extract was not added to the control samples (freshly drawn blood, and blood stored for 21 and 42 days). Results are reported in Table 1.

TABLE 1
Addition of LUKOR to whole blood

| | Day O | | Day 21 | | Day 42 | |
|---|---|---|---|---|---|---|
| | Untreat- | | Untreat- | | Untreat- | |
| Parameter | ed | Treated | ed | Treated | ed | Treated |
| RBC | 5.3 | 5.3 | 4.2 | 5.1 | 3.0 | 5.1 |
| Hb | 12.8 | 12.9 | 8.4 | 12.8 | 6.3 | 12.6 |
| Hct | 32.3% | 32.3% | 20.2% | 33.0% | 15.8% | 32.0% |
| Lysis | 0% | 0% | 10% | 0% | 40% | 0% |
| 45% saline | 0% | 0% | 92% | 0% | 58.4% | 0% |

The effects of LUKOR on Factor VIII and Factor IX clotting activities were determined by adding LUKOR (1:10 dilution) to human plasma and incubating the samples for 1, 2, 5, and 23 hours at 37° C. The samples were frozen and assayed for Factor VIII and Factor IX clotting activity by a one-stage activated prothrombin time (APTT) method using 0.15 M sodium chloride as the control. Denson, Br. J. Haematol., 1973, 24(4):451-461. Samples also were incubated at room temperature and at 4° C. Factor VIII and Factor IX activities of the samples treated with LUKOR (0.076 and 0.389 units/ml respectively) were not different from saline controls (Table 2, % activity remaining).

TABLE 2
Clotting Activity in Treated Samples

| Time | | | | |
|---|---|---|---|---|
| (Hrs) | FVIII/saline | FVIII/LUKOR | FIX/Saline | FIX/LUKOR |
| 0 | 91.81 | 111.11 | 91.13 | 93.01 |
| 1 | 93.92 | 114.29 | 98.77 | 103.06 |
| 2 | 80.11 | 120.91 | 96.95 | 93.83 |
| 5 | 93.5 | 114.29 | 93.51 | 93.68 |
| 23 | 67.95 | 87.45 | 40.14 | 62.03 |

In vivo toxicology of LUKOR was examined using Sprague-Dawley CD albino rats. Five male rats and five female rats were administered 1 ml of LUKOR per day by intravenous injection into the tail vein on 6 occasions (on Days 1, 3, 5, 7, 9 and 28). All animals were examined twice daily for mortality and signs of ill health or reaction to treatment, with a more detailed examination performed weekly. There were no treatment-related clinical signs and the animals did not develop hypersensitivity reactions by the end of the experiment on Day 28. No treatment-related effects on food consumption or weight gain were observed. Blood samples were collected under anesthesia from the orbital sinus, 24 hours following the dose at day 9, and again from the abdominal aorta at necropsy at day 28. All animals were euthanized by exsanguinations from the abdominal aorta following anesthesia. At the termination of the study on Day 28, hematological evaluations were performed on all animals. There were no mortalities, no treatment-related clinical observations, or effects on white cell parameters.

### Example 2

### Cytotoxicity of LUKOR on Cultured Blood Mononuclear Cells

Cultured human blood mononuclear cells were cultured in the presence of varying concentrations of LUKOR for 7 days. Solutions containing different concentrations of LUKOR were prepared by diluting the stock solution of LUKOR (1 mg/mL) 1:4, 1:20, 1:100, and 1:500 with PBS. An equal volume of each dilution of LUKOR was added to cultured cells. Medium was changed at Day 3. The resulting cell counts at each concentration of LUKOR are listed in Table 3. A colorimetric assay was used to assess cytotoxicity. A WST-1 test kit (a tetrazolium compound that is the sodium salt of 4-[3-(4-iodophenyl)-2-(4-nitrophenyl)-2H-5-tetra olio]-1,3-benzene dislocate) was used in this assay (Roche Diagnostics, Indianapolis, Ind.).

TABLE 3
Cytotoxicity of LUKOR

| Dilution (1X) | WST-1 (%) | Cell Count (%) |
|---|---|---|
| 0 | 100 | 100 |
| 4 | 84 | 78.8 |
| 20 | 98 | 106 |
| 100 | 101 | 100 |
| 500 | 104 | 106 |

### Example 3

### Identification of Active Component from LUKOR

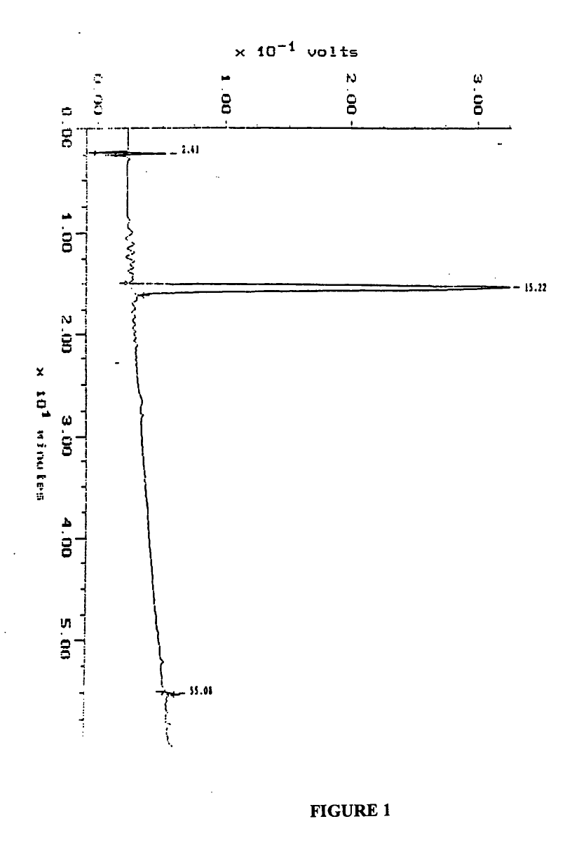

The soluble lysate isolated from PBMCs (LUKOR) was fractionated by HULK as a first step in the identification of the active component. A C18 column (Delta Pak, 15 μm, 300Δ, 0.39×30 cm) with a mobile phase of 0.1% tetrafluoroacetic acid (TFA) in water and a gradient of 0-100% acetonitrile (ACN) was used to separate the components in the cell lysate. One major peak eluting with 30% ACN and two minor peaks at 50% ACN were observed (FIG. 1). The 3 peaks, designated HPLC-1, HPLC-2, and HPLC-3, were collected separately and lyophilized and stored for further characterization by mass spectrometry and NMR.

Mass spectrometry was performed with a VG BioQ triple quadrupole mass spectrometer operating in the positive ion electrospray ionization mode using the following parameters: scan range m/z 100-950 and 35-700; cone voltage 57V to 63V; source temperature 80° C. to 100° C. Calibration was performed with direction injection analysis of CsI prior to LC-MS. A distinct aromatic ring absorbance with a peak maximum at 258 nm was detected. Behavior of the compound was consistent with a small molecular weight compound. Sample related masses of 86, 194, and 288 were identified. The sample related masses of 86 and 194 represented less than 1% of total. It was determined that the sample mass of 288 was composed of carbon, hydrogen, oxygen, and a single nitrogen atom.

Proton NMR also was performed on the extract in a solution of D₂O using a modified Nicolet NT 360 MH3 spectrometer operating with a single 0.5 μsec excitation pulse and a one second re-cycle delay. Signals were detected with shifts between 3.1-3.7 ppm.

The active component of LUKOR was identified as N-glycolylneuraminic acid based on the molecular weight and chemical composition.

### Example 4

### Assessment of Cell Proliferation in Cells Treated with N-glycolylneuraminic Acid

On day 0, cell lines listed in Table 4 were plated into microtiter plates at 850-2000 cells/ well in 100 μL of media. On day 1, N-glycolylneuraminic acid was diluted 2× in medium and from 0 to 200 μM of N-glycolylneuraminic acid or 1000 μM of N-glycolylneuraminic acid were added. The stock solution at 50/250 mM in DMSO then was diluted 1/500 in media, V_{f}=200 μL/well. The cells were incubated for 3 days at 37° C. and 5% CO₂. On day 4, ³[H]-thymidine, diluted 1/100 in media, was added at 25 μL/well/200 μL of medium, resulting in a final concentration of 0.5 μCi per well. On day 5, cells were harvested (18 hours after the addition of the ³[H]-thymidine) onto a glass fiber, and CPM/well were determined. The results are listed in Table 4.

TABLE 4
Treated Cell Lines

| | | Plating | % |
|---|---|---|---|
| Cell | | cpm | Confluency |
| Line | Description | (day 0) | (day 5) |
| Molt-4 | Peripheral blood, acute lymphoblastic | 2000 | 50 |
| | leukemia, human. | | |
| DU-145 | Prostate carcinoma, metastasis | 1200 | 100 |
| | to brain, human | | |
| HSF | Human foreskin fibroblast, | 1000 | 100 |
| | diploid, normal. | | |
| HT1080 | Fibrosarcoma, | 850 | 100 |
| | epithelial-like, human. | | |
| HepG2 | Hepatocellular carcinoma, human. | 2000 | 50 |

### Example 5

### Inhibition of Tumor Necrosis Factor (TNF) with N-Glycolylneuraminic Acid

TNFα production was examined in latently infected with HIV promonocytic U1 cells that were incubated in the presence of N-glycolylneuraminic acid (0.1 to 316 μm). TNFα production was determined by an enzyme linked immunosorbent assay (ELISA) in a competitive format, using a commercially available anti-TNFα antibody (Sigma). Inhibition of TNFα production is reported as percent of saline control in Table 5.

TABLE 5
Inhibition of TNFα Production in U1 Cells

| Conc. of N-Glycolylneuraminic Acid | Inhibition of TNFα Production |
|---|---|
| (μM) | % of Control |
| 0.1 | 0 |
| 0.3 | 0 |
| 10 | 9.8 |
| 30 | 22.6 |
| 100 | 96 |

### Example 6

### Treatment of Breast Cancer

A patient diagnosed with breast cancer with wide spread metastasis was treated with Lukor in addition to conventional therapy. A CAT scan, X rays and lab reports showed great improvement in size of tumors (reduced from 12 cm to 2 cm ) in 4 weeks treatment with Lukor. Cancer antigen (CA) level dropped from 3500 to 715.

Another breast cancer patient had a mastectomy followed by 3 months of treatment with Lukor. The patient's cancer has been in remission for over 3 years.

### Example 7

### Treatment of Prostate Cancer

A patient was diagnosed with prostrate cancer with a prostate antigen (PSA) level of 8.5, and was given one dose of Lupron that gave him intolerable side effects. He was treated with Lukor orally at a dose of 5 mg per day for 21 days, his PSA level dropped to 2.5. He regained lost weight. Further treatment with Lukor for an additional 15 days reduced his PSA level to 0.5. Normal 2.

### Example 8

### Treatment of Ovarian Cancer

A patient was pronounced as terminally ill, with metastases in liver, lungs, stomach. Her liver tumor was reduced and stomach obstruction was relieved in about two weeks upon use of Lukor.

### Example 9

### Treatment of Uterine Cancer

A patient was admitted into the hospital for evaluation of persistent cough and shortness of breath. She was diagnosed with uterine sarcoma with widespread metastases to liver and lungs, with bilateral renal obstruction and chronic heart failure (CHF) left ventricular ejection fraction (LVEF) 25%. She was given Lukor and treated for CHF and released from the hospital with no pulmonary distress. She also received conventional chemotherapy. The 150 day report showed reduction in tumor lesions in all areas, no spread of cancer into other organs. Day 301 report shows 50% reduction in tumor size and return of LVEF to normal (64%).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a human patient having leukemia comprising, administering to said patient a composition comprising,
at least one of a N-glycolylneuraminic acid or a derivative thereof, and
at least one pharmaceutically acceptable excipient, wherein the administration of the composition is at least sufficient to slow progression of the leukemia.

2. The method of claim 1, further comprising the steps of staging the leukemia in the patient prior to administering the composition, and staging the leukemia post-administration of the composition, wherein the administration comprises a dosing regimen of the composition such that the post-administration staging is the same or less severe than prior to administration.

3. The method of claim 1, wherein the leukemia does not progress after administration of the composition.

4. The method of claim 1, wherein the leukemia regresses after administration of the composition.

5. The method of claim 1, further comprising the steps of determining a baseline growth rate of the leukemia prior to administering the composition, and determining a post-administration growth rate of the leukemia, wherein the administration comprises a dosing regimen of the composition such that the post-administration growth rate is less than 97% of the baseline growth rate.

6. The method of claim 1, further comprising the steps of determining a baseline TNFα level prior to administering the composition, and determining a post-administration TNFα level, wherein the administration comprises a dosing regimen of the composition such that the post-administration TNFα level is less than 95% of the baseline TNFα level.

7. The method of claim 1, further comprising the steps of determining a baseline TNFα level prior to administering the composition, and determining a post-administration TNFα level, wherein the administration comprises a dosing regimen of the composition such that the post-administration TNFα level is less than 90% of the baseline TNFα level.

8. The method of claim 1, further comprising the steps of determining a baseline TNFα level prior to administering the composition, and determining a post-administration TNFα level, wherein the administration comprises a dosing regimen of the composition such that the post-administration TNFα level is less than 60% of the baseline TNFα level.

9. The method of claim 1, wherein at least some of the N-glycolylneuraminic acid or derivative thereof is bound, and the composition further comprises a catalytic amount of at least one pharmaceutically acceptable acid or a salt thereof.

10. The method of claim 1, wherein the composition is administered intravenously, subcutaneously, by inhalation, transdermally, or bucally.

11. The method of claim 1, wherein the composition is administered buccally.

12. The method of claim 11, wherein the composition is at least partially dissolved by saliva in the patient's mouth.

13. The method of claim 11, wherein at least some of the N-glycolylneuraminic acid or derivative thereof is bound, and the composition further comprises a catalytic amount of at least one pharmaceutically acceptable acid or a salt thereof, and at least some of the acid or salt thereof is dissolved by saliva in the patient's mouth.

14. The method of claim 1, wherein the dosing regimen comprises administering the composition at least once per day.

15. The method of claim 1, wherein the dosing regimen comprises administering to the patient between about 0.1 mg and 1000 mg of the composition per day.

16. The method of claim 1, wherein the dosing regimen comprises administering to the patient between about 0.2 mg and 100 mg of the composition per day.

17. The method of claim 1, wherein the dosing regimen comprises administering to the patient between about 0.2 mg and 80 mg of the composition per day.

18. The method of claim 1, wherein a TNFα level in the patient is lower in the patient post-administration of the composition than before administration of the composition.

19. The method of claim 1, wherein the leukemia is acute lymphoblastic leukemia, chronic lymphocytic leukemia, acute myelogenous leukemia, or chronic myelogenous leukemia.

20. The method of claim 1, wherein the composition is administered in addition to at least one additional leukemia treatment or a medication to alleviate or reduce a symptom of the leukemia or the additional leukemia treatment.

21. The method of claim 1, wherein the composition comprises between about 0.002 wt % and 20 wt % of the N-glycolylneuraminic acid or the derivative thereof.

22. The method of claim 1, wherein the composition comprises between about 0.1 wt % and 20 wt % of the N-glycolylneuraminic acid or the derivative thereof.

23. The method of claim 1, wherein the composition comprises between about 0.1 wt % and 10 wt % of the N-glycolylneuraminic acid or the derivative thereof.

24. The method of claim 1, wherein the composition comprises N-glycolylneuraminic acid.

25. The method of claim 1, wherein the composition comprises a derivative of N-glycolylneuraminic acid.

26. The method of claim 1, wherein the composition comprises a phosphorylated N-glycolylneuraminic acid.

27. The method of claim 1, wherein the composition comprises a sulfated N-glycolylneuraminic acid.

28. The method of claim 1, wherein composition comprises synthetic N-glycolylneuraminic acid.

29. The method of claim 1, wherein composition comprises N-glycolylneuraminic acid extracted from a biological sample.

30. The method of claim 1, wherein the composition comprises a biological sample comprising the N-glycolylneuraminic acid or the derivative thereof

31. The method of claim 1, wherein the composition is in the form of a tablet, a lozenge, a sucker, a semi-soft candy, a gum, a gel, a paste, a mouthwash, or a film.

32. The method of claim 1, wherein the composition further comprises at least one of a coloring agent, a polypeptide, a lubricant, a coating, a sweetener, a flavoring, an antibacterial agent, a taste modifier, a preservative, a disintegrator, a disintegration-preventor, a binder, an antioxidant, a dietary supplement, an antiblocking agent, an antisticking agent, an absorption promoter, an absorption-adsorption carrier, or a therapeutic agent that is pharmaceutically acceptable.
